(19) United States
(12) Patent Application Publication
(10) Pub. No.: US 2025/0312768 A1
(43) Pub. Date: Oct. 9, 2025

(54) IRON-BASED POWDER FOR OXYGEN REACTANT AND OXYGEN REACTANT

(71) Applicant: JFE STEEL CORPORATION, Chiyoda-ku, Tokyo (JP)

(72) Inventors: Naoki YAMAMOTO, Chiyoda-ku, Tokyo (JP); Kohsuke ASHIZUKA, Chiyoda-ku, Tokyo (JP); Shigeru UNAMI, Chiyoda-ku, Tokyo (JP)

(73) Assignee: JFE STEEL CORPORATION, Chiyoda-ku, Tokyo (JP)

(21) Appl. No.: 18/865,336

(22) PCT Filed: Jun. 2, 2023
(86) PCT No.: PCT/JP2023/020709

§ 371 Date: Nov. 13, 2024

(30) Foreign Application Priority Data
Jul. 11, 2022 (JP) 2022-111434
Feb. 16, 2023 (WO) PCT/JP2023/005564

(51) Int. Cl. B01J 20/20; B01J 20/02

(52) U.S. Cl. CPC B01J 20/20; B01J 20/0229; B01J 2220/42

(57) ABSTRACT

Provided is iron-based powder for an oxygen reactant that has excellent oxygen reactivity. The iron-based powder for an oxygen reactants contains iron powder having an atomic number ratio of oxygen to iron, O/Fe, of 0.30 or less and carbonaceous powder having a C content of 50 mass % or more. The carbonaceous powder content is 0.20 mass % or more and 30.00 mass % or less.

### TECHNICAL FIELD

The present disclosure relates to iron-based powder for an oxygen reactant and an oxygen reactant.

### BACKGROUND

Deoxidizing agents and exothermic agents are known example applications of oxygen reactants that utilize the reaction between iron-based powder and oxygen. Deoxidizing agents are sealed in containers along with preserved items such as foods and medicines to keep the preserved items in a low-oxygen state and suppress quality deterioration due to oxidation, mold growth, and the like. Exothermic agents are widely used as a disposable body warmers to warm the human body and the like. Typically, in such oxygen reactants, activated carbon, sodium chloride, silica powder, wood powder, water, sulfur powder, and the like are added to iron-based powder to further promote the oxygen reaction.

Further, in any application where the reaction rate between iron and oxygen is important, mixing a conductive substance powder other than iron with iron powder has conventionally been considered as a means to control the reaction rate.

For example, JP 2003-117385 A (Patent Literature (PTL) 1) describes a deoxidizing agent using activated iron powder whose iron powder surface is partially covered with an electrically conductive carbonaceous material such as conductive graphite, carbon black, graphite, or activated carbon at 0.3 wt % to 3.0 wt %.

PTL 1 describes that in a so-called free carbon state released from iron powder, there is no effect on the oxygen absorbing property of the deoxidizing agent, and therefore an operation to partially cover the iron powder surface is necessary.

### CITATION LIST

### Patent Literature

PTL 1: JP 2003-117385 A

### SUMMARY

### Technical Problem

In the description of PTL 1, composition of the iron powder itself is not specified. In particular, when the surface of iron powder is oxidized, forming iron ions becomes difficult, making electron emission difficult. Therefore, when the surface of iron powder is oxidized, as is commonly observed, the phenomenon described in PTL 1, in which the iron powder acts as an anode and a carbon substance acts as a cathode to promote an oxidation reaction, is less likely to occur.

It would be helpful to provide an iron-based powder for an oxygen reactant and an oxygen reactant that have excellent oxygen reactivity.

### Solution to Problem

As mentioned above, carbonaceous powders such as graphite powder, coal powder, and activated carbon powder are considered to have no effect on the oxygen absorption properties of an oxygen deoxidizing agent in a free carbon state, and therefore do not function as an oxygen reactant.

Carbonaceous powder as described above has a higher standard electrode potential than iron, and the potential increases further as iron oxidizes, as illustrated by the potential/pH diagram (Pourbaix diagram) of iron.

When iron powder comes into contact with carbonaceous powder that is an electrically conductive powder having a higher potential than iron powder in a corrosion environment such as electrolytic solution, a local battery mechanism occurs in which corrosion current flows from the high-potential carbonaceous powder to the low-potential iron powder, and then returns via the electrolyte solution to the high-potential carbonaceous powder, and then flows back to the iron powder. When such a mechanism occurs, the reaction between the low-potential iron powder and oxygen may be promoted.

Here, the inventors arrived at the idea of adding carbonaceous powder in a free carbon state that does not react easily with oxygen to iron powder, and mixing, in order to accelerate the reaction between the iron-based powder and oxygen, and conducted extensive studies. As a result, the inventors found that there is an appropriate mixing ratio of iron-based powder that exhibits excellent oxygen reactivity.

The present disclosure is based on the above findings, and primary features are as follows.
1. Iron-based powder for an oxygen reactant, the iron-based powder comprising:
iron powder having an atomic number ratio of oxygen to iron, O/Fe, of 0.30 or less; and
carbonaceous powder having a C content of 50 mass % or more, wherein
the carbonaceous powder content is 0.20 mass % or more and 30.00 mass % or less.
2. The iron-based powder for an oxygen reactant according to 1, above, wherein the carbonaceous powder comprises at least one of coal powder or coke powder.
3. The iron-based powder for an oxygen reactant according to 1, above, wherein the carbonaceous powder comprises coal powder and coke powder.
4. The iron-based powder for an oxygen reactant according to any one of 1 to 3, above, wherein the carbonaceous powder comprises at least one of activated carbon powder or carbon black powder.
5. The iron-based powder for an oxygen reactant according to any one

of 1 to 4, above, wherein the carbonaceous powder comprises 0.12 mass % or more of coke powder.
6. The iron-based powder for an oxygen reactant according to any one of 1 to 5, above, wherein the carbonaceous powder comprises coke powder and carbon black powder.
7. The iron-based powder for an oxygen reactant according to 6, above, wherein
the coke powder content is 3.50 mass % or more, and
the carbon black powder content is 6.20 mass % or more.
8. An oxygen reactant using the iron-based powder for an oxygen reactant according to any one of 1 to 7, above.

### Advantageous Effect

According to the present disclosure, iron-based powder for an oxygen reactant and an oxygen reactant that have excellent oxygen reactivity are obtainable.

### DETAILED DESCRIPTION

An embodiment of the present disclosure is described below. The following embodiment is an example that is illustrative of the present disclosure, and the present disclosure is not limited to only the described embodiment.

Reasons why the iron-based powder for an oxygen reactant according to the present disclosure exhibits excellent oxygen reactivity are presumed to be as follows. Carbonaceous powder has a higher potential than iron powder, and therefore when carbonaceous powder and iron powder come into contact in an electrolyte solution, a corrosion current is generated, which promotes the oxidation reaction of the iron powder. Further, the iron-based powder for an oxygen reactant according to the present disclosure has excellent reactivity with oxygen and is therefore suitable for use to make the oxygen reactant according to the present disclosure. Accordingly, the oxygen reactant according to the present disclosure can achieve the same characteristics and effects as the iron-based powder for an oxygen reactant according to the present disclosure.

Here, iron increases in potential as it oxidizes, and therefore iron powder prior to use as an oxygen reactant should be as unoxidized as possible to facilitate a larger potential difference with carbonaceous powder. This results in a larger corrosion current. Therefore, according to the present disclosure, a ratio of oxygen to iron atoms in iron powder of the iron-based powder for an oxygen reactant (hereinafter also referred to as “O/Fe”) needs to be 0.30 or less. In this O/Fe range, the potential difference between the carbonaceous powder and the iron powder in an electrolyte solution becomes large enough to generate an effective amount of corrosion current (sufficient to promote the oxidation reaction of the iron powder). Therefore, in the present disclosure, O/Fe in the iron powder of the iron-based powder for an oxygen reactant is 0.30 or less. A lower limit of O/Fe is not particularly specified and may be 0, but is preferably about 0.15 for industrial purposes. Further, the value of O/Fe is measurable according to a method described below.

The iron powder used in the present disclosure may be produced by water atomization, gas atomization, pulverization, and oxide reduction methods. Further, according to the present disclosure, carbonaceous powder is added to such iron powder. Such carbonaceous powder may be commercially available activated carbon powder, coke powder, carbon black powder, or the like.

Here, the term “iron-based powder” in the present disclosure refers to a metal powder containing 50.0 mass % or more Fe. Further, in addition to the metallic iron (Fe), the iron-based powder may further contain any element such as C, S, O, N, Si, Na, Mg, Ca, or the like. The metallic iron content of the iron-based powder is measurable according to the “metallic iron quantification method” in Japanese Industrial Standard JIS A 5011-2.

The iron-based powder for an oxygen reactant is a mixture of iron powder and carbonaceous powder, where the carbonaceous powder content in the mixture is 0.20 mass % or more and 30.00 mass % or less. When the carbonaceous powder content in the iron-based powder for an oxygen reactant is less than 0.20 mass %, the amount of corrosion current is low and there is no effect of promoting the oxygen reaction of iron powder. On the other hand, the carbonaceous powder itself is less oxidizable and reacts less with oxygen than the iron powder, and therefore when the carbonaceous powder content in the iron-based powder for an oxygen reactant is greater than 30.00 mass %, the oxygen reaction volume of the mixture of the iron powder and the carbonaceous powder becomes lower than that of the iron powder alone (for example, 60 mL/g). From the viewpoint of oxygen reactivity, the carbonaceous powder content in the iron-based powder for an oxygen reactant is preferably 0.50 mass % or more. Further, the carbonaceous powder content is preferably 15.00 mass % or less.

The present disclosure provides the iron-based powder for an oxygen reactant that meets the requirements described above to achieve excellent oxygen reactivity.

The particle size of the iron powder is not particularly limited as long as no handling problems are caused. The particle size in median size (median particle size from cumulative volume frequency) D₅₀ is preferably 1 mm or less. D₅₀ is more preferably 400 μm or less. D₅₀ is even more preferably 200 μm or less. On the other hand, a lower limit of the particle size is preferably about 5 μm in terms of handling. Further, D₅₀ is measurable according to a method described below.

The carbonaceous powder according to the present disclosure is carbonaceous powder having a C content (carbon content) of 50 mass % or more in the carbonaceous powder. When the C content in the carbonaceous powder is less than 50 mass %, the amount of corrosion current is low and there is no effect of promoting the oxygen reaction of iron powder. The C content in the carbonaceous powder is preferably 60 mass % or more. The C content in the carbonaceous powder is more preferably 70 mass % or more. On the other hand, an upper limit is not particularly limited. Although the C content in the carbonaceous powder may be 100 mass %, in economic terms the upper limit of the C content in the carbonaceous powder is preferably about 95 mass %.

The particle size of the carbonaceous powder is not particularly limited as long as no handling problems are caused. The particle size in median size D₅₀ is preferably 100 μm or less. D₅₀ is more preferably 50 μm or less. D₅₀ is even more preferably 30 μm or less. On the other hand, a lower limit of the particle size of the carbonaceous powder is preferably about 5 μm in terms of handling.

The method for measuring the median size D₅₀ of the iron powder and the carbonaceous powder is as follows. The iron powder and the carbonaceous powder to be measured are put into ethanol as a solvent, dispersed by ultrasonic oscillation for 30 s or longer, and the particle size is measured by a laser diffraction and scattering method using a laser diffraction particle size distribution analyzer. That is, volume-based particle size distribution of particles of the iron powder and the carbonaceous powder are respectively measured. The cumulative particle size distribution is calculated from the particle size distribution obtained, and the particle size corresponding to 50% of the sum of the volume of all particles is determined as the median size D₅₀. According to the present disclosure, the median size D₅₀ is used as a representative value for the particle size of the iron powder and the carbonaceous powder, respectively.

### [Method of Calculating O/Fe in Powder]

According to the present disclosure, the method of calculating O/Fe in powder is preferably as follows. A target powder is measured by X-ray diffraction, and obtained diffraction data is analyzed by Rietveld analysis to determine the content of Fe alone, compounds of Fe and O, and other compounds in the powder. The number of Fe and O atoms can be determined from such content values, and therefore the value of O/Fe can be calculated.

### [Production of Iron Powder]

The iron powder used according to the present disclosure is preferably prepared by a water atomizing method or gas atomization, in which water or gas is sprayed onto molten metal, which is then pulverized, cooled and solidified; or by reducing iron oxide (mill scale) generated on steel sheet surfaces during hot rolling of steel material; or by reducing iron ore powder. Further, the prepared powder may be classified and selected or mixed in various ways to adjust into the iron powder according to the present disclosure. To remove oxygen to achieve the O/Fe range described above, deoxidation may be performed at 750° C. or more using carbon such as coke or graphite or hydrogen gas.

### [Carbonaceous Powder]

The carbonaceous powder according to the present disclosure may be a commercial product such as coal powder, activated carbon powder, coke powder, carbon black powder, or the like. For example, in the case of coke powder, production may be as follows. In the production of such coke powder, coal is dry distilled at 1000° C. or more for 10 h or more to remove volatile matter and tar contained in the coal, and then pulverized and classified and selected. Further, recovered powder generated during pulverizing and classifying and selecting may also be suitably used.

### [Production of Iron-Based Powder]

In the production of the iron-based powder for an oxygen reactant, the iron powder and the carbonaceous powder need to be mixed. In the iron-based powder for an oxygen reactant, the mixing of the iron powder and the carbonaceous powder is preferably uniform. Therefore, it is preferable to use a mixing device such as a V-shaped mixer, a double cone mixer, or a conical blender. The above devices and associated mixing conditions may be used according to a publicly known method.

### [Oxygen Reactant]

According to the present disclosure, the iron-based powder for an oxygen reactant may be used to make an oxygen reactant. For example, when the iron-based powder for an oxygen reactant is sealed in a bag described below, the iron-based powder for an oxygen reactant can be made into the oxygen reactant according to the present disclosure. In the oxygen reactant, components other than the iron-based powder for an oxygen reactant can be used without particular restriction as long as they are used in conventionally known oxygen reactants. Examples of such components include a bag of air-permeable packaging material made of non-woven fabric and open-pore polyethylene overlaid, or a bag of air-permeable packaging material made of paper and open-pore polyethylene overlaid.

### Examples

The iron-based powders for an oxygen reactant used in the present examples were prepared by the following procedure. Thirty-nine different iron powders with different O/Fe were prepared by hydrogen reduction of iron ore powder. To prepare each of the iron-based powders for an oxygen reactant, the iron powder, two types of powdered lignite coal (A and B), sub-bituminous coal, activated carbon powder (Shirasagi PHC-14, produced by Osaka Gas Chemicals Co., Ltd.), coke powder made by pulverizing coke prepared by dry distilling coal at 1200° C. for 15 h, and carbon black powder (REGAL 330R, produced by Cabot Corporation) were fed into a V-shaped mixer and mixed. Here, the C content of the lignite coal A was 58.7 mass %. The C content of the lignite coal B was 68.2 mass %. The C content of the sub-bituminous coal was 75.7 mass %. The C content of the activated carbon powder was 89.8 mass %. The C content of the coke powder was 81.7 mass %. The C content of the carbon black powder was 98.1 mass %. The O/Fe of the iron powders was calculated by measuring the content of Fe alone, compounds of Fe and O, and other compounds with an X-ray diffractometer (SmartLab, produced by Rigaku Holdings Corporation).

For the present examples, an oxygen reactivity evaluation of the iron-based powder for an oxygen reactant was performed as follows. To obtain each oxygen reactant, 0.6 g of an aqueous solution with a sodium chloride concentration of 12 mass % was added to 1.5 g of zeolite (Zeofill 1424 #, particle size 1.0 mm to 2.0 mm, produced by Shin Tohoku Chemical Industry Co., Ltd.), then 1.5 g of the iron-based powder for an oxygen reactant was added to the mixture and filled into a bag of an air-permeable packaging material (length 50 mm x width 60 mm). A layered material consisting of non-woven fabric and open-pore polyethylene was used for the air-permeable packaging material. One of each oxygen reactant was sealed, along with 3 L of air, in a gas barrier bag of layered material consisting of nylon/aluminum foil/polyethylene. After the bags were left at 25° C. for 8 h, the oxygen concentration in the bags was measured using a gas chromatograph (GC3210D, produced by GL Sciences Inc.). The oxygen reaction volume was calculated from the difference between the measured oxygen concentration and the oxygen concentration in air, and the oxygen reaction volume per 1 g of the iron-based powder for an oxygen reactant was calculated.

Table 1 lists the results of oxygen reaction volume of the iron-based powders for an oxygen reactant according to each Comparative Example and each Example according to the present disclosure. Further, Table 2 lists the median size (D₅₀) of the iron powders used in the Examples and Comparative Examples. Table 3 lists the median size (D₅₀) of the carbonaceous powders used in the Examples and Comparative Examples. In the following description, the iron-based powder for an oxygen reactant may be referred to simply as iron-based powder.

TABLE 1

| | Iron-based powder | | | | | | | | |
|---|---|---|---|---|---|---|---|---|---|
| | Iron powder | | | | | | | | |
| | Atomic | Carbonaceous powder | | | | | | | Evaluation result |
| | number ratio | | | Sub- | | Activated | Carbon | Total | Oxygen reaction |
| | of oxygen to | Lignite | Lignite | bituminous | Coke | carbon | black | carbonaceous | volume of iron- |
| | iron O/Fe | coal A | coal B | coal | powder | powder | powder | powder | based powder |
| Test No. | (—) | (mass %) | (mass %) | (mass %) | (mass %) | (mass %) | (mass %) | (mass %) | (mL/g) |
| Comparative Example 1 | 0.02 | — | — | — | — | — | — | 0.00 | 58 |
| Comparative Example 2 | 0.32 | — | — | — | 0.25 | — | — | 0.25 | 58 |
| Comparative Example 3 | 0.34 | — | — | — | 29.82 | — | — | 29.82 | 55 |
| Comparative Example 4 | 0.02 | — | — | — | 0.13 | — | — | 0.13 | 53 |
| Comparative Example 5 | 0.28 | — | — | — | 31.03 | — | — | 31.03 | 51 |
| Comparative Example 6 | 0.37 | — | — | — | 0.09 | 0.12 | — | 0.21 | 52 |
| Comparative Example 7 | 0.35 | — | — | — | 15.78 | 12.84 | — | 28.62 | 56 |
| Comparative Example 8 | 0.27 | — | — | — | 0.10 | 0.07 | — | 0.17 | 58 |
| Comparative Example 9 | 0.02 | — | — | — | 16.77 | 13.42 | — | 30.19 | 51 |
| Comparative Example 10 | 0.32 | — | — | — | 0.11 | — | 0.16 | 0.27 | 53 |
| Comparative Example 11 | 0.31 | — | — | — | 11.04 | — | 17.82 | 28.86 | 58 |
| Comparative Example 12 | 0.12 | — | — | — | 0.12 | — | 0.03 | 0.15 | 55 |
| Comparative Example 13 | 0.30 | — | — | — | 5.23 | — | 26.24 | 31.47 | 55 |
| Comparative Example 14 | 0.32 | — | — | — | 0.06 | 0.12 | 0.04 | 0.22 | 56 |
| Comparative Example 15 | 0.38 | — | — | — | 5.21 | 22.67 | 1.78 | 29.66 | 58 |
| Comparative Example 16 | 0.01 | — | — | — | 0.12 | 0.03 | 0.03 | 0.18 | 52 |
| Comparative Example 17 | 0.22 | — | — | — | 7.24 | 17.35 | 5.87 | 30.46 | 56 |
| Example 1 | 0.06 | 0.42 | — | — | — | — | — | 0.42 | 60 |
| Example 2 | 0.06 | 4.23 | — | — | — | — | — | 4.23 | 61 |
| Example 3 | 0.06 | 29.82 | — | — | — | — | — | 29.82 | 62 |
| Example 4 | 0.05 | — | 0.37 | — | — | — | — | 0.37 | 61 |
| Example 5 | 0.05 | — | 15.85 | — | — | — | — | 15.85 | 62 |
| Example 6 | 0.05 | — | 28.47 | — | — | — | — | 28.47 | 62 |
| Example 7 | 0.05 | — | — | 0.47 | — | — | — | 0.47 | 62 |
| Example 8 | 0.05 | — | — | 10.35 | — | — | — | 10.35 | 63 |
| Example 9 | 0.05 | — | — | 27.79 | — | — | — | 27.79 | 63 |
| Example 10 | 0.06 | — | — | — | 28.92 | — | — | 28.92 | 67 |
| Example 11 | 0.06 | — | — | — | 20.35 | — | — | 20.35 | 66 |
| Example 12 | 0.06 | — | — | — | 29.56 | — | — | 29.56 | 67 |
| Example 13 | 0.15 | — | — | — | 0.12 | 0.42 | — | 0.54 | 72 |
| Example 14 | 0.15 | — | — | — | 5.00 | 5.00 | — | 10.00 | 74 |
| Example 15 | 0.15 | — | — | — | 11.00 | 18.50 | — | 29.50 | 75 |
| Example 16 | 0.02 | — | — | — | 6.23 | — | 8.24 | 14.47 | 79 |
| Example 17 | 0.02 | — | — | — | 3.52 | — | 6.24 | 9.76 | 77 |
| Example 18 | 0.02 | — | — | — | 16.57 | — | 12.57 | 29.14 | 79 |
| Example 19 | 0.04 | — | — | — | 0.43 | 0.52 | 0.25 | 1.20 | 81 |
| Example 20 | 0.04 | — | — | — | 16.57 | — | 11.28 | 27.85 | 82 |

* Underlining indicates value outside scope of present disclosure.

TABLE 2

| Test No. | Iron powder D₅₀ (μm) |
|---|---|
| Comparative Example 1 | 95 |
| Comparative Example 2 | 83 |
| Comparative Example 3 | 145 |
| Comparative Example 4 | 64 |
| Comparative Example 5 | 77 |
| Comparative Example 6 | 120 |
| Comparative Example 7 | 85 |
| Comparative Example 8 | 75 |
| Comparative Example 9 | 70 |
| Comparative Example 10 | 92 |
| Comparative Example 11 | 80 |
| Comparative Example 12 | 92 |
| Comparative Example 13 | 88 |
| Comparative Example 14 | 103 |
| Comparative Example 15 | 85 |
| Comparative Example 16 | 82 |
| Comparative Example 17 | 152 |
| Example 1 | 104 |
| Example 2 | 92 |
| Example 3 | 95 |
| Example 4 | 84 |
| Example 5 | 76 |
| Example 6 | 69 |
| Example 7 | 68 |
| Example 8 | 98 |
| Example 9 | 102 |
| Example 10 | 85 |
| Example 11 | 92 |
| Example 12 | 85 |
| Example 13 | 88 |
| Example 14 | 82 |
| Example 15 | 74 |
| Example 16 | 114 |
| Example 17 | 93 |
| Example 18 | 90 |
| Example 19 | 85 |
| Example 20 | 98 |

TABLE 3

| | Carbonaceous powder D₅₀ (μm) |
|---|---|
| Lignite coal A | 65 |
| Lignite coal B | 48 |
| Sub-bituminous coal | 52 |
| Coke powder | 32 |
| Activated carbon powder | 18 |
| Carbon black powder | 10 |

As listed in Table 1, the oxygen reaction volumes of the iron-based powders of Examples 1 to 20, in which the atomic number ratio of oxygen to iron in the iron powder, O/Fe, is 0.30 or more and the carbonaceous powder content having a C content of 50 mass % or more in the powder mixture with the iron powder is 0.20 mass % or more and 30.0 mass % or less and containing at least one of coal powder or coke powder, are greater than that of the iron-based powders of Comparative Examples 1 to 17. Further, the oxygen reaction volumes per 1 g of the iron-based powders are 60 mL/g or more, indicating very good oxygen reaction volume.

Among these, the iron-based powders of Examples 10 to 20, which include coke powder as the carbonaceous powder, all have an oxygen reaction volume of 65 mL/g or more per 1 g of the iron-based powder, indicating that they have superior oxygen reactivity in terms of oxygen reaction volume.

The iron-based powders of Examples 10 to 20 all contain 0.12 mass % or more of coke powder. When the iron-based powder contains 0.12 mass % or more of coke powder, superior oxygen reactivity is seen in terms of oxygen reaction volume.

The iron-based powders of Examples 16 to 20, which include coke powder and carbon black powder, all have an oxygen reaction volume of 77 mL/g or more per 1 g of the iron-based powder, indicating that they have excellent oxygen reactivity in terms of oxygen reaction volume.

Among the iron-based powders of Examples 16 to 20 containing coke powder and carbon black powder, the iron-based powder of Example 19, which further contains activated carbon powder, has an excellent oxygen reaction volume of 81 mL/g per 1 g of the iron-based powder, even though the carbonaceous powder content is only 1.20 mass %.

The iron-based powders of Examples 16 to 18 and 20, which contain only coke powder and carbon black powder as carbonaceous powder, indicate excellent oxygen reactivity when containing 3.50 mass % or more of coke powder and 6.20 mass % or more of carbon black powder.

The results of Comparative Examples 5, 9, 13, and 17 indicate that iron-based powder does not have excellent oxygen reactivity when the total amount of carbonaceous powder exceeds 30.00 mass %. In particular, even when the iron-based powder contains coke powder and carbon black powder as carbonaceous powder, the iron-based powder does not have excellent oxygen reactivity when the total amount of carbonaceous powder exceeds 30.00 mass %, as in Comparative Examples 13 and 17. Therefore, the total carbonaceous powder needs to be 30.00 mass % or less.

A comparison of the iron-based powder of Example 19 containing coke powder, activated carbon powder, and carbon black powder as carbonaceous powder, the iron-based powders of Examples 16 to 18 and 20 containing only coke powder and carbon black powder as carbonaceous powder, the iron-based powders of Examples 13 to 15 containing only coke powder and activated carbon powder as carbonaceous powder, the iron-based powders of Examples 10 to 12 and Comparative Examples 4 and 5, which contain only coke powder as carbonaceous powder, further illustrates the following points.

When the iron-based powder contains only coke powder, the iron-based powder has excellent reactivity when the coke powder content as carbonaceous powder is 0.20 mass % or more and 30.00 mass % or less (Examples 10 to 12). However, when the coke powder content as carbonaceous powder is less than 0.20 mass % (Comparative Example 4) or when the coke powder content exceeds 30.00 mass % (Comparative Example 5), the iron-based powder does not have excellent reactivity.

Although the iron-based powder has very good reactivity even when the iron-based powder contains only coke powder in a defined amount as described above, rather than increasing or decreasing the coke powder content in the iron-based powder, the coexistence of another carbonaceous powder such as activated carbon or carbon black powder in addition to coke powder (Examples 13 to 20) results in superior oxygen reactivity. In particular, when the iron-based powder contains at least coke powder and carbon black powder (Examples 16 to 20), the iron-based powder has excellent oxygen reactivity.

The iron-based powders of Examples 1 to 9, in which the coal powder content as carbonaceous powder is 0.20 mass % or more and 30.00 mass % or less, have a stable oxygen reaction volume per 1 g of iron-based powder of 60 mL/g or more and 63 mL/g or less, with very little variation. When the coal powder content as carbonaceous powder is 0.20 mass % or more and 30.00 5 mass % or less, very good oxygen reactivity and stable oxygen reactivity without variation is found in terms of oxygen reaction volume.

In this way, the iron-based powder for an oxygen reactant and the oxygen reactant that have excellent oxygen reactivity can be provided.

### INDUSTRIAL APPLICABILITY

The present disclosure is applicable to iron-based powders for an oxygen reactant and oxygen reactants.

1. Iron-based powder for an oxygen reactant, the iron-based powder comprising:
iron powder having an atomic number ratio of oxygen to iron, O/Fe, of 0.30 or less; and
carbonaceous powder having a C content of 50 mass % or more, wherein
the carbonaceous powder content is 0.20 mass % or more and 30.00 mass % or less.

2. The iron-based powder for an oxygen reactant according to claim 1, wherein the carbonaceous powder comprises at least one of coal powder or coke powder.

3. The iron-based powder for an oxygen reactant according to claim 1, wherein the carbonaceous powder comprises coal powder and coke powder.

4. The iron-based powder for an oxygen reactant according to claim 1, wherein the carbonaceous powder comprises at least one of activated carbon powder or carbon black powder.

5. The iron-based powder for an oxygen reactant according to claim 1, wherein the carbonaceous powder comprises 0.12 mass % or more of coke powder.

6. The iron-based powder for an oxygen reactant according to claim 1, wherein the carbonaceous powder comprises coke powder and carbon black powder.

7. The iron-based powder for an oxygen reactant according to claim 6, wherein
the coke powder content is 3.50 mass % or more, and
the carbon black powder content is 6.20 mass % or more.

8. An oxygen reactant using the iron-based powder for an oxygen reactant according to claim 1.

9. An oxygen reactant using the iron-based powder for an oxygen reactant according to claim 2.

10. An oxygen reactant using the iron-based powder for an oxygen reactant according to claim 3.

11. An oxygen reactant using the iron-based powder for an oxygen reactant according to claim 4.

12. An oxygen reactant using the iron-based powder for an oxygen reactant according to claim 5.

13. An oxygen reactant using the iron-based powder for an oxygen reactant according to claim 6.

14. An oxygen reactant using the iron-based powder for an oxygen reactant according to claim 7.
